SEQUENCE LISTING (17 sequences)

SEQ ID NO: 1 - DNA, 2517 bp, Artificial Sequence
  cDNA sequence encoding Homo sapiens TLR4 isoform A precursor.
atgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatc

SEQ ID NO: 2 - protein, 839 aa, Artificial Sequence
  Amino acid sequence of Homo sapiens TLR4 isoform A precursor
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 3 - DNA, 1824 bp, Artificial Sequence
  cDNA sequence encoding the extracellular domain of Homo sapiens TLR4 isoform A
gaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcgacaccttcagataagcagggcatgcctgtgctgagtttgaatatcacctgtcagatgaataag

SEQ ID NO: 4 - protein, 608 aa, Artificial Sequence
  Amino acid sequence of the extracellular domain of Homo sapiens TLR4 isoform A
ESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNK

SEQ ID NO: 5 - DNA, 2529 bp, Artificial Sequence
  cDNA sequence encoding extracellular domain of Homo sapiens TLR4 isoform A fused at its carboxy terminus to an IgG1 antibody Fc domain
gaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcgacaccttcagataagcagggcatgcctgtgctgagtttgaatatcacctgtcagatgaataagggatccgctagcgagaacctgtacttccagagcgctagctgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 843 aa, Artificial Sequence
  Amino acid sequence of extracellular domain of Homo sapiens TLR4 isoform A fused at its carboxy terminus to an IgG1 antibody Fc domain
ESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKGSASENLYFQSASCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 2505 bp, Artificial Sequence
  cDNA sequence encoding Mus musculus TLR4 precursor
atgatgcctccctggctcctggctaggactctgatcatggcactgttcttctcctgcctgacaccaggaagcttgaatccctgcatagaggtagttcctaatattacctaccaatgcatggatcagaaactcagcaaagtccctgatgacattccttcttcaaccaagaacatagatctgagcttcaaccccttgaagatcttaaaaagctatagcttctccaatttttcagaacttcagtggctggatttatccaggtgtgaaattgaaacaattgaagacaaggcatggcatggcttacaccacctctcaaacttgatactgacaggaaaccctatccagagtttttccccaggaagtttctctggactaacaagtttagagaatctggtggctgtggagacaaaattggcctctctagaaagcttccctattggacagcttataaccttaaagaaactcaatgtggctcacaattttatacattcctgtaagttacctgcatatttttccaatctgacgaacctagtacatgtggatctttcttataactatattcaaactattactgtcaacgacttacagtttctacgtgaaaatccacaagtcaatctctctttagacatgtctttgaacccaattgacttcattcaagaccaagcctttcagggaattaagctccatgaactgactctaagaggtaattttaatagctcaaatataatgaaaacttgccttcaaaacctggctggtttacacgtccatcggttgatcttgggagaatttaaagatgaaaggaatctggaaatttttgaaccctctatcatggaaggactatgtgatgtgaccattgatgagttcaggttaacatatacaaatgatttttcagatgatattgttaagttccattgcttggcgaatgtttctgcaatgtctctggcaggtgtatctataaaatatctagaagatgttcctaaacatttcaaatggcaatccttatcaatcattagatgtcaacttaagcagtttccaactctggatctaccctttcttaaaagtttgactttaactatgaacaaagggtctatcagttttaaaaaagtggccctaccaagtctcagctatctagatcttagtagaaatgcactgagctttagtggttgctgttcttattctgatttgggaacaaacagcctgagacacttagacctcagcttcaatggtgccatcattatgagtgccaatttcatgggtctagaagagctgcagcacctggattttcagcactctactttaaaaagggtcacagaattctcagcgttcttatcccttgaaaagctactttaccttgacatctcttatactaacaccaaaattgacttcgatggtatatttcttggcttgaccagtctcaacacattaaaaatggctggcaattctttcaaagacaacaccctttcaaatgtctttgcaaacacaacaaacttgacattcctggatctttctaaatgtcaattggaacaaatatcttggggggtatttgacaccctccatagacttcaattattaaatatgagtcacaacaatctattgtttttggattcatcccattataaccagctgtattccctcagcactcttgattgcagtttcaatcgcatagagacatctaaaggaatactgcaacattttccaaagagtctagccttcttcaatcttactaacaattctgttgcttgtatatgtgaacatcagaaattcctgcagtgggtcaaggaacagaagcagttcttggtgaatgttgaacaaatgacatgtgcaacacctgtagagatgaatacctccttagtgttggattttaataattctacctgttatatgtacaagacaatcatcagtgtgtcagtggtcagtgtgattgtggtatccactgtagcatttctgatataccacttctattttcacctgatacttattgctggctgtaaaaagtacagcagaggagaaagcatctatgatgcatttgtgatctactcgagtcagaatgaggactgggtgagaaatgagctggtaaagaatttagaagaaggagtgccccgctttcacctctgccttcactacagagactttattcctggtgtagccattgctgccaacatcatccaggaaggcttccacaagagccggaaggttattgtggtagtgtctagacactttattcagagccgttggtgtatctttgaatatgagattgctcaaacatggcagtttctgagcagccgctctggcatcatcttcattgtccttgagaaggttgagaagtccctgctgaggcagcaggtggaattgtatcgccttcttagcagaaacacctacctggaatgggaggacaatcctctggggaggcacatcttctggagaagacttaaaaatgccctattggatggaaaagcctcgaatcctgagcaaacagcagaggaagaacaagaaacggcaacttggacc

SEQ ID NO: 8 - protein, 835 aa, Artificial Sequence
  Amino acid sequence of Mus musculus TLR4 precursor
MMPPWLLARTLIMALFFSCLTPGSLNPCIEVVPNITYQCMDQKLSKVPDDIPSSTKNIDLSFNPLKILKSYSFSNFSELQWLDLSRCEIETIEDKAWHGLHHLSNLILTGNPIQSFSPGSFSGLTSLENLVAVETKLASLESFPIGQLITLKKLNVAHNFIHSCKLPAYFSNLTNLVHVDLSYNYIQTITVNDLQFLRENPQVNLSLDMSLNPIDFIQDQAFQGIKLHELTLRGNFNSSNIMKTCLQNLAGLHVHRLILGEFKDERNLEIFEPSIMEGLCDVTIDEFRLTYTNDFSDDIVKFHCLANVSAMSLAGVSIKYLEDVPKHFKWQSLSIIRCQLKQFPTLDLPFLKSLTLTMNKGSISFKKVALPSLSYLDLSRNALSFSGCCSYSDLGTNSLRHLDLSFNGAIIMSANFMGLEELQHLDFQHSTLKRVTEFSAFLSLEKLLYLDISYTNTKIDFDGIFLGLTSLNTLKMAGNSFKDNTLSNVFANTTNLTFLDLSKCQLEQISWGVFDTLHRLQLLNMSHNNLLFLDSSHYNQLYSLSTLDCSFNRIETSKGILQHFPKSLAFFNLTNNSVACICEHQKFLQWVKEQKQFLVNVEQMTCATPVEMNTSLVLDFNNSTCYMYKTIISVSVVSVIVVSTVAFLIYHFYFHLILIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 9 - DNA, 1821 bp, Artificial Sequence
  cDNA sequence encoding the extracellular domain ofMus musculus TLR4
ggcagcctgaacccgtgcattgaagtggtgccgaacattacctatcagtgcatggatcagaaactgagcaaagtgccggatgatattccgagcagcaccaaaaacattgatctgagctttaacccgctgaaaattctgaaaagctatagctttagcaactttagcgaactgcagtggctggatctgagccgctgcgaaattgaaaccattgaagataaagcgtggcatggcctgcatcatctgagcaacctgattctgaccggcaacccgattcagagctttagcccgggcagctttagcggcctgaccagcctggaaaacctggtggcggtggaaaccaaactggcgagcctggaaagctttccgattggccagctgattaccctgaaaaaactgaacgtggcgcataactttattcatagctgcaaactgccggcgtattttagcaacctgaccaacctggtgcatgtggatctgagctataactatattcagaccattaccgtgaacgatctgcagtttctgcgcgaaaacccgcaggtgaacctgagcctggatattagcctgaacccgattgattttattcaggatcaggcgtttcagggcattaaactgcatgaactgaccctgcgcggcaactttaacagcagcaacattatgaaaacctgcctgcagaacctggcgggcctgcatgtgcatcgcctgattctgggcgaatttaaagatgaacgcaacctggaaatttttgaaccgagcattatggaaggcctgtgcgatgtgaccattgatgaatttcgcctgacccataccaacgattttagcgatgatattgtgaaatttcattgcctggcgaacgtgagcgcgatgagcctggcgggcgtgagcattaaatatctggaagatgtgccgaaacattttaaatggcagagcctgagcattattcgctgccagctgaaacagtttccgaccctggatctgccgtttctgaaaagcctgaccctgaccatgaacaaaggcagcattagctttaaaaaagtggcgctgccgagcctgagctatctggatctgagccgcaacgcgctgagctttagcggctgctgcagctatagcgatctgggcaccaacagcctgcgccatctggatctgagctttaacggcgcgattattatgagcgcgaactttatgggcctggaagaactgcagcatctggattttcagcatagcaccctgaaacgcgtgaccgaatttagcgcgtttctgagcctggaaaaactgctgtatctggatattagctataccaacaccaaaattgattttgatggcatttttctgggcctgaccagcctgaacaccctgaaaatggcgggcaacagctttaaagataacaccctgagcaacgtgtttgcgaacaccaccaacctgacctttctggatctgagcaaatgccagctggaacagattagctggggcgtgtttgataccctgcatcgcctgcagctgctgaacatgagccataacaacctgctgtttctggatagcagccattataaccagctgtatagcctgagcaccctggattgcagctttaaccgcattgaaaccagcaaaggcattctgcagcattttccgaaaagcctggcgttttttaacctgaccaacaacagcgtggcgtgcatttgcgaacatcagaaatttctgcagtgggtgaaagatcagaaacagtttctggtgaacgtggaacagatgacctgcgcgaccccggtggaaatgaacaccagcctggtgctggattttaacaacagcacctgctatatgtataaa

SEQ ID NO: 10 - protein, 607 aa, Artificial Sequence
  Amino acid sequence of the extracellular domain ofMus musculus TLR4
GSLNPCIEVVPNITYQCMDQKLSKVPDDIPSSTKNIDLSFNPLKILKSYSFSNFSELQWLDLSRCEIETIEDKAWHGLHHLSNLILTGNPIQSFSPGSFSGLTSLENLVAVETKLASLESFPIGQLITLKKLNVAHNFIHSCKLPAYFSNLTNLVHVDLSYNYIQTITVNDLQFLRENPQVNLSLDISLNPIDFIQDQAFQGIKLHELTLRGNFNSSNIMKTCLQNLAGLHVHRLILGEFKDERNLEIFEPSIMEGLCDVTIDEFRLTHTNDFSDDIVKFHCLANVSAMSLAGVSIKYLEDVPKHFKWQSLSIIRCQLKQFPTLDLPFLKSLTLTMNKGSISFKKVALPSLSYLDLSRNALSFSGCCSYSDLGTNSLRHLDLSFNGAIIMSANFMGLEELQHLDFQHSTLKRVTEFSAFLSLEKLLYLDISYTNTKIDFDGIFLGLTSLNTLKMAGNSFKDNTLSNVFANTTNLTFLDLSKCQLEQISWGVFDTLHRLQLLNMSHNNLLFLDSSHYNQLYSLSTLDCSFNRIETSKGILQHFPKSLAFFNLTNNSVACICEHQKFLQWVKDQKQFLVNVEQMTCATPVEMNTSLVLDFNNSTCYMYK

SEQ ID NO: 11 - DNA, 480 bp, Artificial Sequence
  cDNA sequence encoding Homo sapiens MD2 precursor
atgttaccatttctgtttttttccaccctgttttcttccatatttactgaagctcagaagcagtattgggtctgcaactcatccgatgcaagtatttcatacacctactgtgataaaatgcaatacccaatttcaattaatgttaacccctgtatagaattgaaaggatccaaaggattattgcacattttctacattccaaggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaat

SEQ ID NO: 12 - protein, 160 aa, Artificial Sequence
  Amino acid sequence of Homo sapiens MD2 precursor
MLPFLFFSTLFSSIFTEAQKQYWVCNSSDASISYTYCDKMQYPISINVNPCIELKGSKGLLHIFYIPRRDLKQLYFNLYITVNTMNLPKRKEVICRGSDDDYSFCRALKGETVNTTISFSFKGIKFSKGKYKCVVEAISGSPEEMLFCLEFVILHQPNSN

SEQ ID NO: 13 - DNA, 480 bp, Artificial Sequence
  cDNA sequence encoding Mus musculus MD2 precursor
atgttgccatttattctcttttcgacgctgctttctcccatattgactgaatctgagaagcaacagtggttctgcaactcctccgatgcaattatttcctacagttattgtgatcacttgaaattccctatttcaattagttctgaaccctgcataagactgaggggaaccaatggatttgtgcatgttgagttcattccaagaggaaacttaaaatatttatatttcaacctattcatcagtgtcaactccatagagttgccgaagcgtaaggaagttctgtgccatggacatgatgatgactattctttttgcagagctctgaaaggagagactgtgaatacatcaataccattctctttcgagggaatactatttcctaagggccattacagatgtgttgcagaagctattgctggggatactgaagaaaagctcttctgtttgaatttcaccatcattcaccgccgtgatgtcaat

SEQ ID NO: 14 - protein, 160 aa, Artificial Sequence
  Amino acid sequence of Mus musculus MD2 precursor
MLPFILFSTLLSPILTESEKQQWFCNSSDAIISYSYCDHLKFPISISSEPCIRLRGTNGFVHVEFIPRGNLKYLYFNLFISVNSIELPKRKEVLCHGHDDDYSFCRALKGETVNTSIPFSFEGILFPKGHYRCVAEAIAGDTEEKLFCLNFTIIHRRDVN

SEQ ID NO: 15 - DNA, 2607 bp, Artificial Sequence
  cDNA sequence encoding HGH (Human Growth Hormone) signal sequence fused at its carboxy terminus to the extracellular domain of Homo sapiens TLR4 isoform A which is in turn fused at its carboxy terminus to an IgG1 antibody Fc domain
atggctacaggctcccggacgtccctgctcctggcttttggcctgctctgcctgccctggcttcaagagggatccgccgaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcgacaccttcagataagcagggcatgcctgtgctgagtttgaatatcacctgtcagatgaataagggatccgctagcgagaacctgtacttccagagcgctagctgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 869 aa, Artificial Sequence [sequence omitted]

SEQ ID NO: 17 - DNA, 13744 bp, Artificial Sequence
  Expression vector encoding HGH (Human Growth Hormone) signal sequence fused at its carboxy terminus to the extracellular domain of Homo sapiens TLR4 isoform A which is in turn fused at its carboxy terminus to an IgG1 antibody Fc domain
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacctccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagcttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgtctagaagctgggtaccagctgctagcaagcttgctagcggccgccaccatggctacaggctcccggacgtccctgctcctggcttttggcctgctctgcctgccctggcttcaagagggatccgccgaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcgacaccttcagataagcagggcatgcctgtgctgagtttgaatatcacctgtcagatgaataagggatccgctagcgagaacctgtacttccagagcgctagctgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgcttttccaaaccaccctccttttttgcgccctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccat

(19) United States
(12) Patent Application Publication
(10) Pub. No.: US 2009/0136509 A1
(43) Pub. Date: May 28, 2009

(54) Use of Toll-Like Receptor 4 Antagonists for the Treatment or Prevention of Osteoarthritic Conditions

(76) Inventors: Simon Blake, Radnor, PA (US); Vedrana Stojanovic-Susulic, Radnor, PA (US); Mark Ware, San Diego, CA (US); Linda Wu, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/262,699

(22) Filed: Oct. 31, 2008

Related U.S. Application Data
(60) Provisional application No. 60/984,044, filed on Oct. 31, 2007.

(51) Int. Cl. A61K 39/395

(52) U.S. Cl. 424/139.1; 424/172.1

(57) ABSTRACT

Methods for treating or preventing osteoarthric conditions using Toll-Like Receptor 4 (TLR4) antagonists are disclosed.

### CROSS-REFERENCE TO RELATED APPLICATIONS

This application claims the benefit of U.S. Provisional Application Ser. No. 60/984,044, filed 31 Oct. 2007, the entire contents of which is incorporated herein by reference.

### FIELD OF THE INVENTION

The present invention relates to therapeutic and prophylactic uses of Toll-Like Receptor 4 (TLR4) antagonists.

### BACKGROUND OF THE INVENTION

Osteoarthritis is a joint disease characterized by degeneration of the articular cartilage, excessive bone growth at the joint margins, and changes in the synovial membranes that produce the lubricating fluids which surround the joints (e.g., knee, hip, elbow).

Osteoarthritis represents a significant challenge in health care. Osteoarthritis can be painful and debilitating. Advanced osteoarthritis may require surgeries such as joint replacements or other types of medical intervention. In many individuals symptoms of osteoarthritis begin to appear at middle age, but by age 70 the majority of adults of both genders will be diagnosed with osteoarthritis (Beers and Berkow, Eds. The Merck Manual, 17^{th} Edition, Centennial Edition (2003)). In the United States alone more than 20,000,000 people annually are affected by osteoarthritis (Goldring and Goldring, 213 J. Cell Physiol. 626 (2007)).

In many cases, osteoarthritis is believed to progress, in part, by a mechanism involving cycles of cytokine mediated inflammation, cartilage degradation, and cartilage producing chondrocyte cell death. In other cases, mechanical injury or defects in cartilage production or maintenance can initiate the onset of “osteoarthritis.” Chondrocytes are a cell type that produces and maintain cartilage and are believed to be the cell type responsible for the initiation of osteoarthritis.

Toll Like Receptor 4 (TLR4) forms a receptor complex with the accessory proteins MD2 and CD14 that is activated by exogenous ligands such as lipopolysaccharide (LPS) which binds the MD2 component; TLR4 may also be activated by endogenous ligands (Vistin et al., 175 J. Immunol. 6465 (2005)). Activated TLR4 initiates pro-inflammatory cytokine release and TLR4 activity is believed to play a role in the immune response to infection by gram-negative bacteria which produce LPS (Vistin et al., 175 J. Immunol. 6465 (2005)).

TLR4s, however, may also have important roles in other biological pathways. For example, TLR4 has been shown to be expressed at elevated levels in osteoarthitic cartilage lesions and LPS activation of TLR4 has been shown to increase production of cartilage degradation products by chondrocytes (Kim et al., 54 Arthritis Rheum. 2152 (2006)). Subsequent work with transgenic mice (Mus musculus) in which the TLR4 gene has been inactivated indicates that this LPS induced cartilage degradation product release is mediated by TLR4 (Bobacz et al., 56 Arthritis Rheum. 1880 (2007)).

Thus, a need exists to understand the role of TLR4 in osteoarthritis and exploit this role to develop agents, such as antagonists, and therapies that effectively treat or prevent osteoarthritic conditions.

### BRIEF DESCRIPTION OF THE DRAWINGS

FIG. 1 shows hTLR4A transcript levels are increased in articular cartilage and synoviocytes from human osteoarthritis patients relative to articular cartilage and synoviocytes from non-osteoarthritic individuals.

FIG. 2 shows MD2 transcript levels are increased in articular cartilage but not in synoviocytes from human osteoarthritis patients relative to articular cartilage and synoviocytes from non-osteoarthritic individuals.

FIG. 3 shows CD14 transcript levels are increased in articular cartilage and synoviocytes from human osteoarthritis patients relative to articular cartilage and synoviocytes from non-osteoarthritic individuals.

FIG. 4 shows S-GAG synthesis in lipopolysaccahride (LPS), TLR4/MD2 complex binding antagonist mAb (MTS510), or LPS and MTS510 treated articular cartilage from wild-type mice (Mus musculus) as a percentage of GAG synthesis in untreated articular cartilage from wild-type mice.

FIG. 5 shows S-GAG synthesis in LPS, TLR4/MD2 complex binding antagonist mAb (MTS510), or LPS and MTS510 treated articular cartilage from mTLR4 knockout mice (Mus musculus) as a percentage of GAG synthesis in untreated articular cartilage from mTLR4 knockout mice.

FIG. 6 shows TNF-alpha secretion in LPS, TLR4/MD2 complex binding antagonist mAb (MTS510), or LPS and MTS510 treated articular cartilage from wild-type and mTLR4 knockout mice (Mus musculus).

FIG. 7 shows IL-1alpha secretion in LPS, TLR4/MD2 complex binding antagonist mAb (MTS510), or LPS and MTS510 treated articular cartilage from wild-type and mTLR4 knockout mice (Mus musculus).

FIG. 8 shows IL-1beta secretion in LPS, TLR4/MD2 complex binding antagonist mAb (MTS510), or LPS and MTS510 treated articular cartilage from wild-type and mTLR4 knockout mice (Mus musculus).

FIG. 9 shows GM-CSF secretion in LPS, TLR4/MD2 complex binding antagonist mAb (MTS510), or LPS and MTS510 treated articular cartilage from wild-type and mTLR4 knockout mice (Mus musculus).

FIG. 10 shows RANTES secretion in LPS, TLR4/MD2 complex binding antagonist mAb (MTS510), or LPS and MTS510 treated articular cartilage from wild-type and mTLR4 knockout mice (Mus musculus).

FIG. 11 shows IL-10 secretion in LPS, TLR4/MD2 complex binding antagonist mAb (MTS510), or LPS and MTS510 treated articular cartilage from wild-type and mTLR4 knockout mice (Mus musculus).

FIG. 12 shows KC12 secretion in LPS, TLR4/MD2 complex binding antagonist mAb (MTS510), or LPS and MTS510 treated articular cartilage from wild-type and mTLR4 knockout mice (Mus musculus).

FIG. 13 shows MCP1 secretion in LPS, TLR4/MD2 complex binding antagonist mAb (MTS510), or LPS and MTS510 treated articular cartilage from wild-type and mTLR4 knockout mice (Mus musculus).

FIG. 14 shows IL-6 secretion in LPS, TLR4/MD2 complex binding antagonist mAb (MTS510), or LPS and MTS510 treated articular cartilage from wild-type and mTLR4 knockout mice (Mus musculus).

FIG. 15 shows IP-10 secretion in LPS, TLR4/MD2 complex binding antagonist mAb (MTS510), or LPS and MTS510 treated articular cartilage from wild-type and mTLR4 knockout mice (Mus musculus).

FIG. 16 shows G-CSF secretion in LPS, TLR4/MD2 complex binding antagonist mAb (MTS510), or LPS and MTS510 treated articular cartilage from wild-type and mTLR4 knockout mice (Mus musculus).

FIG. 17 shows MIP-1alpha secretion in LPS, TLR4/MD2 complex binding antagonist mAb (MTS510), or LPS and MTS510 treated articular cartilage from wild-type and mTLR4 knockout mice (Mus musculus).

FIG. 18 shows S-GAG synthesis in LPS, LPS and polymixin B, LPS and the TLR4 antagonist TLR4-ECD, TLR4-ECD alone, IL-1alpha (IL-1a), or TNFalpha (TNFa) treated human chondrocytes as a percentage of GAG synthesis in untreated human chondrocytes.

FIG. 19 shows S-GAG synthesis in LPS, LPS and the TLR4 antagonist TLR4-ECD, TLR4-ECD alone, TNFalpha (TNFa), IL-1alpha (IL1a), IGF1 (Insulin-like Growth Factor 1), LPS and IGF1, IgG1, or LPS and IgG1 treated human chondrocytes from osteoarthritic patients as a percentage of GAG synthesis in untreated human chondrocytes from osteoarthritic patients.

### SUMMARY OF THE INVENTION

One aspect of the invention is a method of treating an osteoarthritic condition comprising administering a therapeutically effective amount of a Toll Like Receptor 4 (TLR4) antagonist to a patient in need thereof for a time sufficient to treat the osteoarthritic condition.

Another aspect of the invention is a method of preventing an osteoarthritic condition comprising administering a therapeutically effective amount of a TLR4 antagonist to a patient in need thereof for a time sufficient to prevent the osteoarthritic condition.

### DETAILED DESCRIPTION OF THE INVENTION

All publications, including but not limited to patents and patent applications, cited in this specification are herein incorporated by reference as though fully set forth.

The term “antagonist” as used herein means a molecule that partially or completely inhibits, by any mechanism, an effect of another molecule such as a receptor. As used herein, a “TLR4 antagonist” or a compound “reactive with TLR4” describes a molecule that is capable of, directly or indirectly, substantially counteracting, reducing or inhibiting TLR4 biological activity or TLR4 receptor activation. Such antagonists may be, for example, small organic molecules, peptide chains, antibodies, antibody fragments, MIMETIBODY™ peptide chains or polynucleotides. Such antagonists may, for example, disrupt the activity of TLR4 by preventing activation or formation of functional complexes comprising TLR4 (e.g. by disrupting MD2 homolog or CD14 homolog activity). The amino acid sequences shown in SEQ ID NO: 12 and SEQ ID NO: 14 are repectively those of Homo sapiens (human) MD2 and Mus musculus (mouse) MD2.

Examples of specific TLR4 antagonists include mAb MTS510, antagonists such as TLR4-ECD which comprises the extracellular domain of a hTLR4A fused to an Fc domain (SEQ ID NO: 2) and others. mAb MTS510 is a monoclonal rat antibody of the IgG2a isotype which binds Mus musculus (mouse) TLR4 and is capable of binding mTLR4 complexed with MD2 as well as inhibiting TLR4 activity. mAb MTS510 is produced by a clone designated MTS510 and is suitable for lyophylization. mAb MTS510 can be obtained from Invivo Gen (San Diego, Calif.) or eBioscience, Inc. (San Diego, Calif.). TLR4-ECD type constructs such as those comprising the amino acid sequence shown in SEQ ID NO: 4 and SEQ ID NO: 10 can also inhibit TLR4 activity and are believed to antagonize TLR4 by inhibiting the interaction of MD2 with TLR4 thus preventing the LPS binding MD2 peptide chain from activating TLR4. The amino acid sequences shown in SEQ ID NO: 6 and SEQ ID NO: 16 are specific examples of such TLR4-ECD construct.

TLR4 antagonists useful in the methods of the invention may also be nucleic acid molecules. Such nucleic acid molecules may be interfering nucleic acid molecules such as short interfering RNAs or antisense molecules that are TLR4 antagonists. Alternatively, polynucleotide molecules such as double and single stranded plasmid DNA vectors, artificial chromosomes, or linear nucleic acids or other vectors that encode a TLR4 antagonist (e.g. peptide chain or RNA), or function as a TLR4 antagonist, may be used in the methods of the invention to administer a TLR4 antagonist to a patient.

The term “antibodies” as used herein is meant in a broad sense and includes immunoglobulin or antibody molecules including polyclonal antibodies, monoclonal antibodies including murine, human, humanized and chimeric monoclonal antibodies and antibody fragments.

In general, antibodies are proteins or polypeptides that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄.

The term “antibody fragments” means a portion of an intact antibody, generally the antigen binding or variable region of the intact antibody. Examples of antibody fragments include Fab, Fab′, F(ab′)₂ and Fv fragments, diabodies, single chain antibody molecules and multispecific antibodies formed from at least two intact antibodies.

The term “antigen” as used herein means any molecule that has the ability to generate antibodies either directly or indirectly. Included within the definition of “antigen” is a protein-encoding nucleic acid.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

CDRs provide the majority of contact residues for the binding of the antibody to the antigen or epitope. CDRs of interest useful in this invention are derived from donor antibody variable heavy and light chain sequences, and include analogs of the naturally occurring CDRs, which analogs also share or retain the same antigen binding specificity and/or neutralizing ability as the donor antibody from which they were derived.

The term “homolog” as used herein means protein sequences having between 75% and 100% sequence identity to a reference sequence. For example, homologs of the mature form of the Homo sapiens MD-2 protein would include those peptide chains that have between 75% and 100% sequence identity to amino acid residues 17 to 160 of SEQ ID NO: 12. Percent identity between two peptide chains can be determined by pair wise alignment using the default settings of the AlignX module of Vector NTI v.9.0.0 (Invitrogen Corp., Carslbad, Calif.).

The term “in combination with” as used herein means that the described agents can be administered to an animal together in a mixture, concurrently as single agents or sequentially as single agents in any order.

The term “inflammatory condition” as used herein means a localized response to cellular injury that is mediated in part by the activity of cytokines, chemokines, or inflammatory cells (e.g., neutrophils, monocytes and lymphocytes) which is characterized in most instances by pain, redness, swelling and loss of tissue function.

The term “MIMETIBODY™ peptide chain” as used herein means a protein having the generic formula (I):

(V1-Pep-Lk-V2-Hg-C_{H}2-C_{H}3)(t) (I)

where V1 is a portion of an N-terminus of an immunoglobulin variable region, Pep is a polypeptide that binds to cell surface TLR4, Lk is a polypeptide or chemical linkage, V2 is a portion of a C-terminus of an immunoglobulin variable region, Hg is a portion of an immunoglobulin hinge region, C_{H}² is an immunoglobulin heavy chain C_{H}2 constant region and C_{H}3 is an immunoglobulin heavy chain C_{H}3 constant region and t is independently an integer of 1 to 10. A MIMETIBODY™ peptide chain can mimic properties and functions of different types of immunoglobulin molecules such as IgG1, IgG2, IgG3, IgG4, IgA, IgM, IgD and IgE dependent on the heavy chain constant domain amino acid sequence present in the construct. In some MIMETIBODY™ peptide chain embodiments, V1 may be absent. A MIMETIBODY™ peptide chain antagonist useful in the present invention affects TLR4 biological activity through binding to cell surface TLR4.

The term “monoclonal antibody” (mAb) as used herein means an antibody (or antibody fragment) obtained from a population of substantially homogeneous antibodies. Monoclonal antibodies are highly specific, typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256:495-497 (1975). Chimeric mAbs containing a light chain and heavy chain variable region derived from a donor antibody (typically murine) in association with light and heavy chain constant regions derived from an acceptor antibody (typically another mammalian species such as human) can be prepared by the method disclosed in U.S. Pat. No. 4,816,567. Humanized mAbs having CDRs derived from a non-human donor immunoglobulin (typically murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl. Acad. Sci. (USA), 86:10029-10032 (1989) and Hodgson et al., Bio/Technology, 9:421 (1991).

Exemplary human framework sequences useful for humanization are disclosed at, e.g., www.ncbi.nlm.nih.gov/entrez/query.fcgi; www.ncbi.nih.gov/igblast; www.atcc.org/phage/hdb.html; www.mrc-cpe.cam.ac.uk/ALIGNMENTS.php; www.kabatdatabase.com/top.html; ftp.ncbi.nih.gov/repository/kabat; www.sciquest.com; www.abcam.com; www.antibodyresource.com/onlinecomp.html; www.public.iastate.edu/˜pedro/research_tools.html; www.whfreeman.com/immunology/CH05/kuby05.htm; www.hhmi.org/grants/lectures/1996/vlab; www.path.cam.ac.uk/˜mrc7/mikeimages.html; mcb.harvard.edu/BioLinks/Immunology.html; www.immunologylink.com; pathbox.wust1.edu/˜hcenter/index.html; www.appliedbiosystems.com; www.nal.usda.gov/awic/pubs/antibody; www.m.ehime-u.ac.jp/˜yasuhito/Elisa.html; www.biodesign.com; www.cancerresearchuk.org; www.biotech.ufl.edu; www.isac-net.org; baserv.uci.kun.n1/˜jraats/links1.html; www.recab.uni-hd.de/immuno.bme.nwu.edu; www.mrc-cpe.cam.ac.uk; www.ibt.unam.mx/vir/V_mice.html; http://www.bioinf.org.uk/abs; antibody.bath.ac.uk; www.unizh.ch; www.cryst.bbk.ac.uk/˜ubcg07s; www.nimr.mrc.ac.uk/CC/ccaewg/ccaewg.html; www.path.cam.ac.uk/˜mrc7/humanisation/TAHHP.html; www.ibt.unam.mx/vir/structure/stat_aim.html; www.biosci.missouri.edu/smithgp/index.html; www.jerini.de; and Kabat et al., Sequences of Proteins of Immunological Interest, U.S. Dept. Health (1987), each entirely incorporated herein by reference.

Fully human mAbs lacking any non-human sequences can be prepared from human immunoglobulin transgenic mice by techniques referenced in, e.g., Lonberg et al., Nature 368:856-859 (1994); Fishwild et al., Nature Biotechnology 14:845-851 (1996) and Mendez et al., Nature Genetics 15:146-156 (1997). Human mAbs can also be prepared and optimized from phage display libraries by techniques referenced in, e.g., Knappik et al., J. Mol. Biol. 296:57-86 (2000) and Krebs et al., J. Immunol. Meth. 254:67-84 (2001).

The terms “osteoarthritic condition” or “osteoarthritis” as used herein means a joint disease characterized by degeneration of the articular cartilage, hypertrophy of bone at the joint margins, and changes in the synovial membrane. In many cases “osteoarthritis” is believed to progress, in part, by a mechanism involving cycles of cytokine mediated inflammation, cartilage degradation, and cartilage producing chondrocyte cell death. In other cases, mechanical injury or defects in cartilage production or maintenance can initiate the onset of “osteoarthritis.”

The term “patient” means an animal belonging to any genus for which treatment of an osteoarthritic condition or prevention of an osteoarthritic condition is indicated.

The term “peptide chain” means a molecule that comprises at least two amino acid residues linked by a peptide bond to form a chain. Large peptide chains of more than 50 amino acids may be referred to as “polypeptides” or “proteins.” Small peptide chains of less than 50 amino acids may be referred to as “peptides.”

In the methods of the invention a “therapeutically effective amount” of a TLR4 antagonist means those doses that, in a given individual patient, produce a response that results in improvement and treatment of one or more symptoms of an ostearthritic condition (e.g. inflammatory cytokine levels). Alternatively, in the methods of the invention a “therapeutically effective amount” of a TLR4 antagonist means those doses that, in a particular individual patient, prevent one or more symptoms of an osteoarthritic condition in an individual such as, for example, an individual pre-disposed to osteoarthritis (e.g. due to mechanical injury to a joint and cartilage or due to defects in cartilage production and maintenance). Therapeutically effective amounts, or doses, appropriate for an individual patient can be readily determined using routine clinical techniques well known by those of skill in the art (e.g. dose response plots).

The term “TLR4” means a peptide chain comprising an amino acid sequence with at least 60% identity to residues 24 to 631 of the amino acid sequence shown in SEQ ID NO: 2 or a complex of peptide chains (e.g. MD2 and CD14) comprising such a peptide chain. SEQ ID NO: 2 shows the amino acid sequence of the Homo sapiens (human) TLR4 isoform A precursor (hTLR4A). Percent identity between two peptide chains can be determined by pair wise alignment using the default settings of the AlignX module of Vector NTI v.9.0.0 (Invitrogen Corp., Carslbad, Calif.).

The term “TLR4 biological activity” or “TLR4 receptor activation” as used herein refers to any activities occurring as a result of ligand binding to TLR4 or complexes comprising a TLR4.

The term “extracellular domain of a TLR4” means a peptide chain comprising an amino acid sequence with at least 60% identity to residues 24 to 631 of the amino acid sequence shown in SEQ ID NO: 2.

Several other sequences relevant to different aspects of the invention are disclosed. These are the sequences shown in SEQ ID NOs 1, 5, 7, 8, 9, 11, 13, 15, and 17. SEQ ID NO: 1 shows a cDNA sequence encoding Homo sapiens TLR4 isoform A precursor. SEQ ID NO: 5 shows a cDNA sequence encoding the extracellular domain of Homo sapiens TLR4 isoform A fused at its carboxy terminus to an IgG1 antibody Fc domain. SEQ ID NO: 7 shows a cDNA sequence encoding Mus musculus TLR4 precursor. SEQ ID NO: 8 shows the amino acid sequence of a Mus musculus TLR4 precursor. SEQ ID NO: 9 shows a cDNA sequence encoding the extracellular domain of Mus musculus TLR4. SEQ ID NO: 11 shows a cDNA sequence encoding Homo sapiens MD2 precursor. SEQ ID NO: 13 shows a cDNA sequence encoding Mus musculus MD2 precursor. SEQ ID NO: 15 shows a cDNA sequence encoding an HGH (Human Growth Hormone) signal sequence fused at its carboxy terminus to the extracellular domain of Homo sapiens TLR4 isoform A which is in turn fused at its carboxy terminus to an IgG1 antibody Fc domain. SEQ ID NO: 17 shows the nucleic acid sequence of an expression vector encoding an HGH (Human Growth Hormone) signal sequence fused at its carboxy terminus to the extracellular domain of Homo sapiens TLR4 isoform A which is in turn fused at its carboxy terminus to an IgG1 antibody Fc domain.

One aspect of the invention is a method of treating an osteoarthritic condition comprising administering a therapeutically effective amount of a Toll Like Receptor 4 (TLR4) antagonist to a patient in need thereof for a time sufficient to treat the osteoarthritic condition.

Another aspect of the invention is a method of preventing an osteoarthritic condition comprising administering a therapeutically effective amount of a TLR4 antagonist to a patient in need thereof for a time sufficient to prevent the osteoarthritic condition.

The TLR4 antagonists useful in the methods of the invention may have the properties of binding a TLR4 receptor and inhibiting TLR4 receptor-mediated signaling. Exemplary mechanisms by which TLR4 signaling may be inhibited by such antagonists include inhibition of kinase activity, transcript reduction or receptor antagonism. Use of other antagonists capable of inhibiting TLR4 receptor-mediated signaling by other mechanisms are also useful in the methods of the invention.

The methods of the invention may be used to treat an animal patient belonging to any genus. Examples of such animals include humans, mice, birds, reptiles, and fish. Without wishing to be bound by any particular theory, it is believed that the therapeutic benefit of TLR4 antagonists will be due to the ability of such antagonists to inhibit the secretion of pro-inflammatory chemokines and cytokines involved in inflammatory conditions.

Amounts of a given TLR4 antagonist sufficient to treat a given inflammatory condition can be readily determined. In the method of the invention the TLR4 antagonist may be administered singly or in combination with at least one other molecule. Such additional molecules may be other TLR4 antagonist molecules or molecules with a therapeutic benefit not mediated by TLR4 receptor signaling. Antibiotics, antivirals, other immunomodulators, other anti-inflammatory agents, leukotriene antagonists, β2 agonists and muscarinic receptor antagonists are examples of such additional molecules.

The mode of administration for therapeutic use of the antagonists of the invention may be any suitable route that delivers the agent to the host. The proteins, antibodies, antibody fragments and MIMETIBODY™ peptide chains and pharmaceutical compositions of these agents are particularly useful for parenteral administration, i.e., intrarticularly, subcutaneously, intramuscularly, intradermally, intravenously or intranasally.

Antagonists useful in the methods of the invention may be prepared as pharmaceutical compositions containing an effective amount of the antagonist as an active ingredient in a pharmaceutically acceptable carrier. An aqueous suspension or solution containing the antagonist, preferably buffered at physiological pH, in a form ready for injection is preferred. The compositions for parenteral administration will commonly comprise a solution of the antagonist of the invention or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well-known sterilization techniques (e.g., filtration). The compositions may contain pharmaceutically acceptable auxiliary substances as required to approximate physiological conditions such as pH adjusting and buffering agents, etc. The concentration of the antagonist of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition useful in the methods of the invention for intramuscular injection could be prepared to contain 1 mL sterile buffered water, and between about 1 ng to about 100 mg, e.g. about 50 ng to about 30 mg or more preferably, about 5 mg to about 25 mg, of an TLR4 antagonist. Similarly, a pharmaceutical composition of the invention for intravenous infusion could be made up to contain about 250 ml of sterile Ringer's solution, and about 1 mg to about 30 mg and preferably 5 mg to about 25 mg of an TLR4 antagonist of the invention. Actual methods for preparing parenterally administrable compositions are well known and are described in more detail in, for example, “Remington's Pharmaceutical Science”, 15th ed., Mack Publishing Company, Easton, Pa. Doses of TLR4 antagonists such as mAbs (e.g. MTS510) or TLR4-ECD may be between about 0.01 mg per kg of animal body weight or 5 mg per kg of animal body weight.

The TLR4 antagonists useful in the methods of the invention, when in a pharmaceutical preparation, can be present in unit dose forms. The appropriate therapeutically effective amount, or dose, can be determined readily by those of skill in the art. A determined dose may, if necessary, be repeated at appropriate time intervals selected as appropriate by a physician during the treatment period.

The peptide chain TLR4 antagonists useful in the methods of the invention can be lyophilized for storage and reconstituted in a suitable carrier prior to use. This technique has been shown to be effective with conventional immunoglobulins and protein preparations and art-known lyophilization and reconstitution techniques can be employed.

In some embodiments of the methods of the invention the TLR4 antagonist is an isolated antibody reactive with TLR4. An antibody is reactive with a TLR4 when, for example, it specifically binds a given TLR4 peptide chain (e.g. Homo sapiens TLR4 isoform A) or a complex comprising a TLR4. The binding of an antagonist, such as a antibody reactive with TLR4, is specific for a given peptide chain when such binding can be used to detect the presence of a first peptide chain (e.g. Homo sapiens TLR4 isoform A), but not a second non-homologous peptide chain (e.g. albumin). This specific binding can be used to distinguish the two peptide chains from each other. Specific binding can be assayed using conventional techniques such as ELISAs and Western blots as well as other techniques well known in the art.

Exemplary antibody antagonists may be antibodies of the IgG, IgD, IgGa or IgM isotypes. Additionally, such antagonist antibodies can be post-translationally modified by processes such as glycosylation, isomerization, aglycosylation or non-naturally occurring covalent modification such as the addition of polyethylene glycol moieties (pegylation) and lipidation. Such modifications may occur in vivo or in vitro. Fully human, humanized and affinity-matured antibody molecules or antibody fragments are useful in the methods of the invention as are MIMETIBODY™ peptide chains, fusion proteins and chimeric proteins.

The antibody antagonists useful in the methods of the invention may specifically bind a TLR4 or complexes comprising a TLR4 with a K_{d} less than or equal to about 10⁻⁷, 10⁻⁸, 10⁻⁹, 10⁻¹⁰, 10⁻¹¹ or 10⁻¹² M. The affinity of a given molecule for a TLR4 receptor or complex comprising a TLR4 can be determined experimentally using any suitable method. Such methods may utilize Biacore or KinExA instrumentation, ELISA or competitive binding assays known to those skilled in the art.

Antibody antagonist molecules binding a given TLR4 homolog with a desired affinity can be selected from libraries of protein variants or fragments by techniques including antibody affinity maturation and other art-recognized techniques suitable for non-antibody molecules.

In some embodiments of the methods of the invention the TLR4 antagonist is an isolated antibody reactive with TLR4 and having the antigen binding ability of the monoclonal antibody MTS510. A isolated antibody reactive with TLR4 has the antigen binding ability of mAb MTS510 when such an isolated antibody competes with mAb MTS510 for binding of a given TLR4 molecule in standard competitive binding assays. Such assays include competitive binding ELISA assays, for example. Those skilled in the art will also recognize other methods appropriate for detecting competition for antigen binding between two antibodies.

TLR4 antagonist antibodies useful in the methods of the invention can further comprise human framework regions selected for their homology to the rat heavy chain amino acid sequence and to the rat light chain amino acid sequence of mAb MTS510.

In some embodiments of the methods of the invention the TLR4 antagonist comprises the extracellular domain of a TLR4.

In some embodiments of the methods of the invention the TLR4 antagonist is a peptide chain comprising the human TLR4 isoform A extracellular domain amino acid sequence shown in SEQ ID NO: 4.

In some embodiments of the methods of the invention the TLR4 antagonist is a peptide chain comprising the mouse TLR4 isoform A extracellular domain amino acid sequence shown in SEQ ID NO: 10.

The present invention will now be described with reference to the following specific, non-limiting examples.

### EXAMPLE 1

### TLR4, MD2, and CD14 Transcript Levels are Increased in Articular Cartilage and Synoviocytes from Human Osteoarthritis Patients

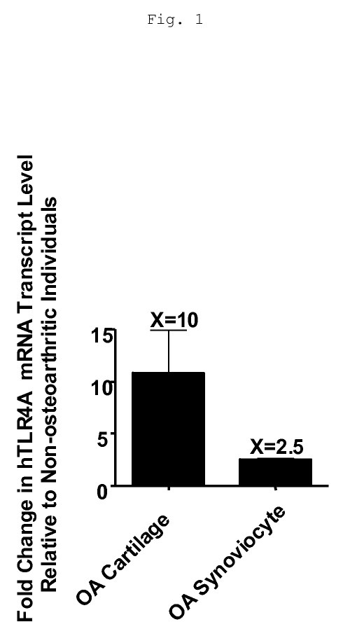

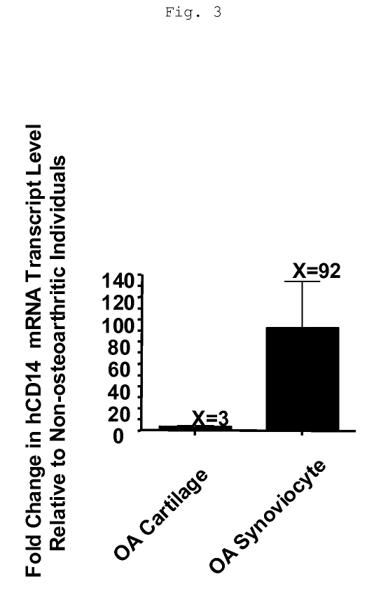

Homo Sapiens TLR4 Isoform A (hTLR4A) and CD14 transcript levels are increased in articular cartilage and synoviocytes from human osteoarthritis patients relative to articular cartilage and synoviocytes from non-osteoarthritic individuals (FIG. 1 and FIG. 3).

hTLR4A, MD2, and CD14 transcript levels in total RNA extracted from the articular cartilage and synoviocytes of human osteoarthritis patients and non-osteoarthritic individuals was measured by real time-PCR (RT-PCR). Total RNA was extracted from samples using TriZOl™ (Invitrogen Corp., Carlsbad, Calif.) and isolated using the RNEasy Mini Kit (Qiagen Inc., Valencia, Calif.). Isolated RNA was then pooled as necessary.

cDNAs were prepared from each RNA pool using the Omniscript™ kit (Qiagen Inc., Valencia, Calif.) according to the manufacturer's instructions. 100 ng of cDNA was amplified using TaqMan™ custom Low Density Array (LDA) Cards (Applied Biosystems, Foster City, Calif.) as directed by the manufacturer. Primer Express™ software (Applied Biosystems) was used to design the probe and primer combinations. TaqMan™ RT-PCR (Applied Biosystems) was then performed in a 384 well format using ABI PRISM™ 7000HT instrumentation (Applied Biosystems) as directed by the manufacturer.

Data collection and transcript quantization in the early exponential phase of PCR was performed with the ABI PRISM™ 7000HT instrumentation and associated software. Individual transcript levels were normalized against transcript levels for 18S ribosomal RNA. Data are expressed as mean fold change in mRNA transcript levels in articular cartilage and synoviocytes from human osteoarthritis patients relative to non-osteoarthritic individuals. Data represents RNA samples from four donors (N=4).

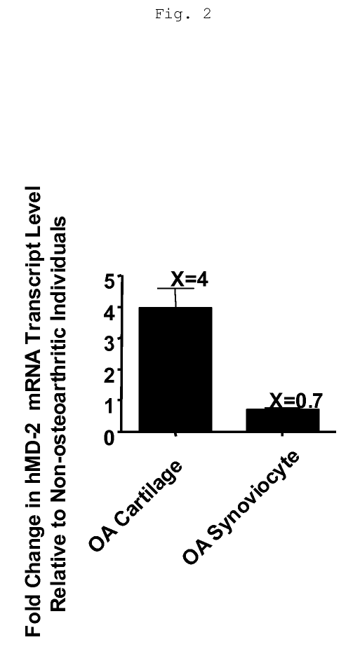

The data indicate that hTLR4A and CD14 transcript levels are increased in cartilage and synoviocytes from human osteoarthritis patients relative to non-osteoarthritic individuals (FIG. 1 and FIG. 3). MD2 transcript levels are increased in cartilage as shown in FIG. 2. hTLR4A, MD2, and CD14 form a complex which mediates TLR4 signaling in response to activation by ligands such as LPS and other signals. Furthermore, TLR4 activation can increase pro-inflammatory cytokine release (see Example 3 below). Consequently, the results here indicate that TLR4 activation and associated inflammatory responses may play an important role in the occurrence of osteoarthritic conditions.

### EXAMPLE 2

### Lipopolysaccahride (LPS) Mediated Decreases in the Synthesis of the Articular Cartilage Component Sulfated Glycosaminoglycan (S-GAG) is TLR4 Dependent and Can Be Reversed by a TLR4 Antagonist mAb

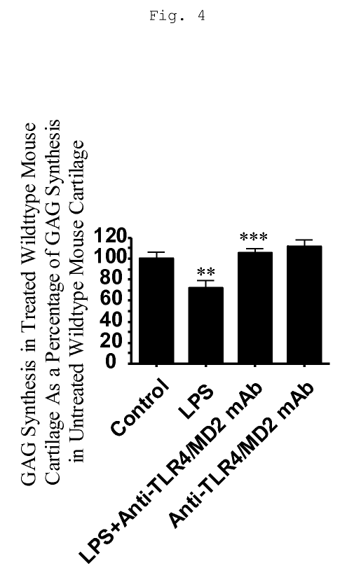

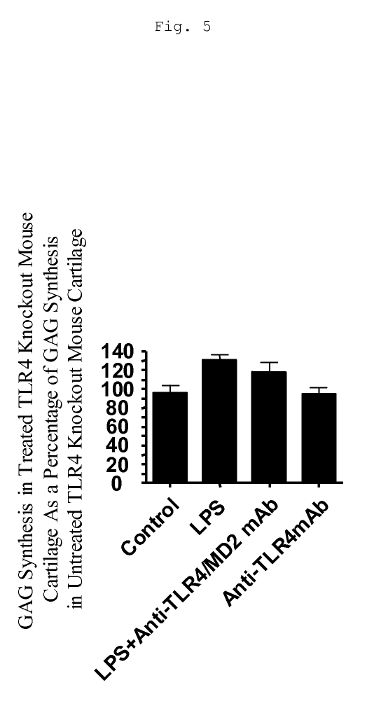

Lipopolysaccahride (LPS) mediated decreases in the synthesis of the articular cartilage component S-GAG is TLR4 dependent and occurs in wild-type mice, but not TLR4 knock-out mice. LPS is an agonist ligand for TLR4 receptors and activates TLR4 mediated signaling. As seen in FIG. 4 and FIG. 5, the synthesis of S-GAG in wild-type mouse articular cartilage explants is decreased by LPS treatment, while LPS treatment of articular cartilage explants from TLR4 knock-out mice does not decrease S-GAG synthesis relative to controls. Importantly, treatment of wild-type mouse articular cartilage explants with LPS and the TLR4/MD2 complex binding antagonist mAb (MTS510) resulted in increased S-GAG synthesis relative to explants treated with LPS alone (FIG. 4).

For these experiments, articular cartilage explants from the femoral heads of 5 week old wild-type mice (Mus musculus) or TLR4 knock-out mice were removed. TLR4 knock-out mice are mice in which the gene encoding mTLR4 has been inactivated. Cartilage explants were prepared and maintained using standard methods. Cartilage explants were incubated in 200 μL of explant culture medium for 3 to 5 days at which time spent media was removed and replaced with fresh media at experiment Day 0. “Control” explants were untreated explants from wild-type (FIG. 4) or TLR4 knock-out mice (FIG. 5). “LPS” treated explants from wild-type (FIG. 4) or TLR4 knock-out mice (FIG. 5) were treated with LPS at a concentration of 10 ng/ml in the media for three days starting at Day 2. “Anti-TLR4/MD2 mAb” treated explants from wild-type (FIG. 4) or TLR4 knock-out mice (FIG. 5) were treated starting at Day 0 with the TLR4/MD2 complex binding antagonist mAb MTS510 (Invivo Gen or eBioscience, Inc., San Diego, Calif.) at a concentration in the media of 20 μg/ml. “LPS+Anti-TLR4/MD2 mAb” treated explants from wild-type (FIG. 4) or TLR4 knock-out mice (FIG. 5) were treated starting at Day 0 with the TLR4/MD2 complex binding antagonist mAb MTS510 at a concentration in the media of 20 μg/ml, followed by treatment on Day 2 with LPS at a concentration of 10 ng/ml in the media for three days. On Day 5 explants were ³⁵S labeled overnight and ³⁵S incorporation into S-GAG (sulfated glycosaminoglycan) was then determined as described by Bobacz et al. 56 Arthritis. Rheum. 1880 (2007).

The results here (FIG. 4 and FIG. 5) indicate that antagonists of the activity of TLR4 receptor complexes, such as mab MTS510, can treat and prevent cartilage degradation in conditions such as osteoarthritis. Data in FIG. 4 and FIG. 5 are presented as mean −/+standard deviation with N=4. In FIG. 3 “**”=P<0.05 versus “Control” and “****”=P<0.001 versus “LPS.”

### EXAMPLE 3

### LPS Induced Pro-Inflammatory Cytokine Release from Articular Cartilage Is Dependent on TLR4 Activity

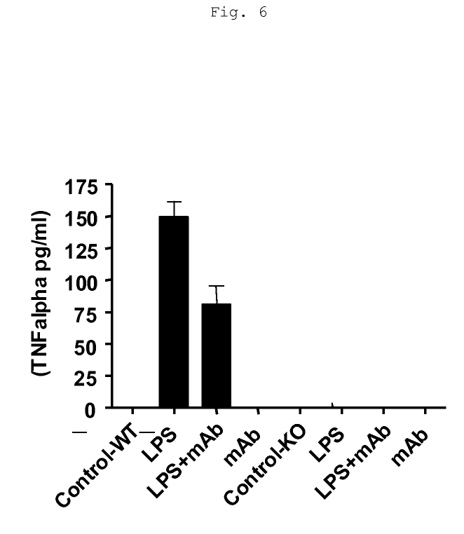

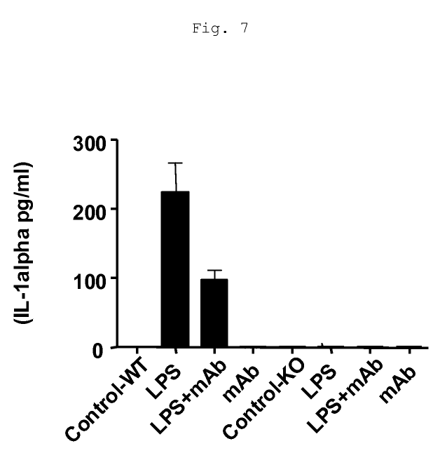

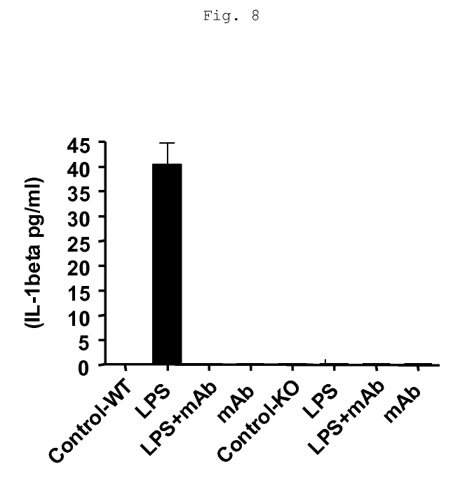

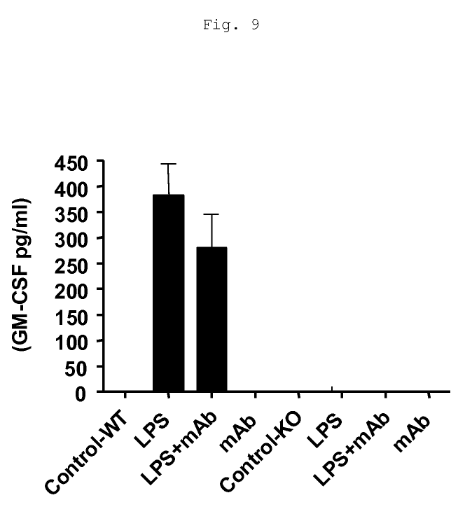

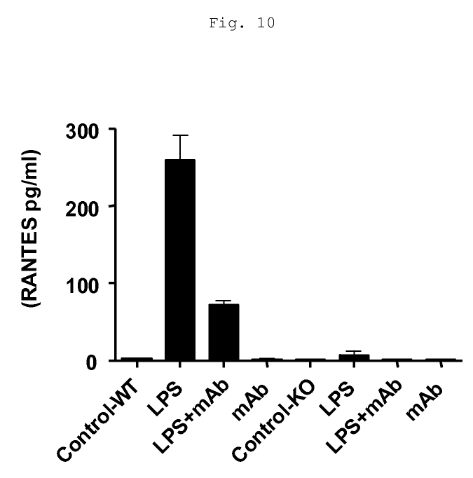

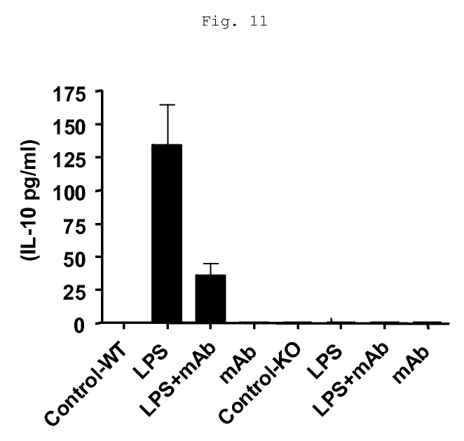

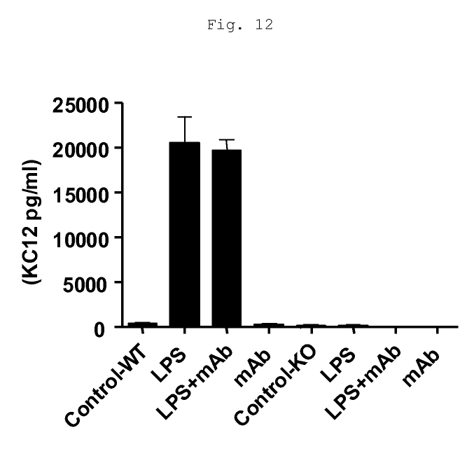

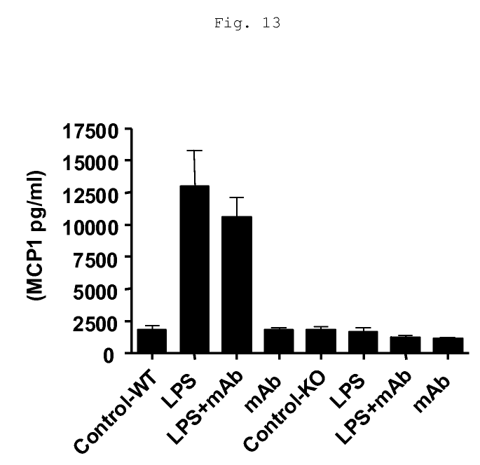

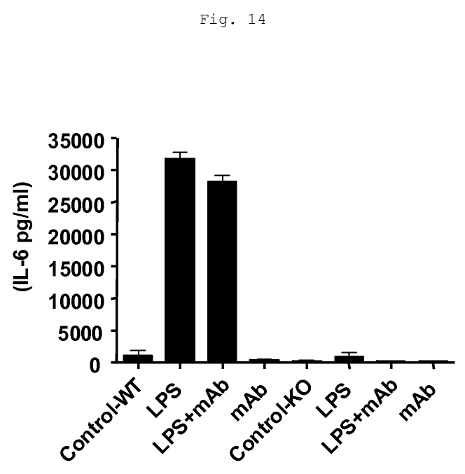

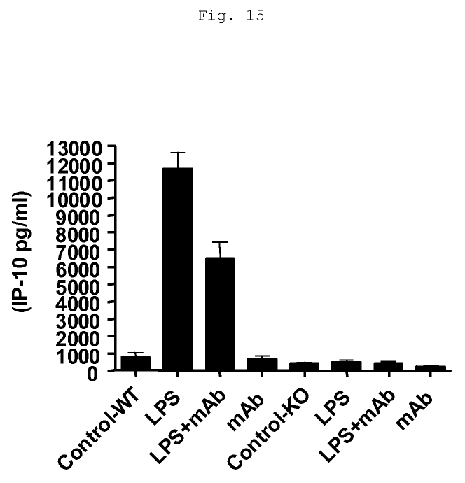

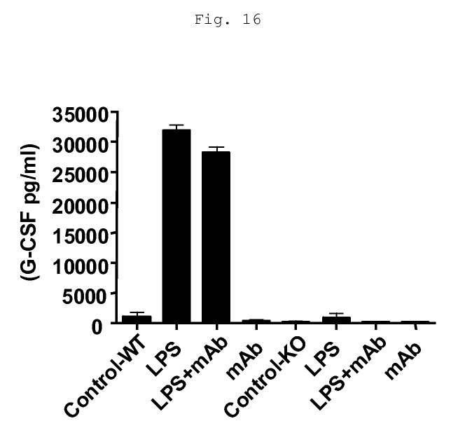

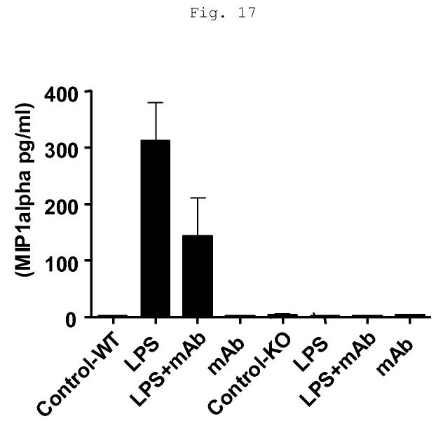

LPS induced release of the pro-inflammatory cytokines TNF-alpha, IL-1alpha, IL-1beta, GM-CSF, RANTES, IL-10, KC12, MCP1, IL-6, IP-10, G-CSF, and MIP-1alpha from articular cartilage is dependent on TLR4 activity (see FIG. 6, FIG. 7, FIG. 8, FIG. 9, FIG. 10, FIG. 11, FIG. 12, FIG. 13, FIG. 14, FIG. 15, FIG. 16, and FIG. 17). Blocking TLR4 activity with the TLR4 antagonist mAb MTS510, or by knocking out the TLR4 gene in mice, prevented or decreased the release of these pro-inflammatory cytokines (see FIG. 6, FIG. 7, FIG. 8, FIG. 9, FIG. 10, FIG. 11, FIG. 12, FIG. 13, FIG. 14, FIG. 15, FIG. 16, and FIG. 17).

For these experiments articular cartilage explants from wild-type (“WT”) and TLR4 knock-out (“KO”) mice were prepared and treated with LPS, as described in Example 2 above. “Control” explants were untreated explants from wild-type or TLR4 knock-out mice. “LPS” treated explants from wild-type or TLR4 knock-out mice were treated with LPS at a concentration of 10 ng/ml in the media for three days starting at Day 2. “mAb” treated explants from wild-type or TLR4 knock-out mice were treated starting at Day 0 with the TLR4/MD2 complex binding antagonist mAb MTS510 at a concentration in the media of 20 μg/ml. “LPS+mAb” treated explants from wild-type or TLR4 knock-out mice were treated starting at Day 0 with the TLR4/MD2 complex binding antagonist mAb MTS510 at a concentration in the media of 20 μg/ml, followed by treatment on Day 2 with LPS at a concentration of 10 ng/ml in the media for three days. Cytokine levels in articular cartilage explant cell culture supernatant media were then measured using LUMINEX® instrumentation (LUMINEX® Corp., Austin, Tex.) TNF-alpha, IL-1alpha, IL-1beta, GM-CSF, RANTES, IL-10, KC12, MCP1, IL-6, IP-10, G-CSF, and MIP-1alpha specific mAb conjugated beads as appropriate. LUMINEX® assays for each cytokine were performed as directed by the manufacturer.

The results here (see FIG. 6, FIG. 7, FIG. 8, FIG. 9, FIG. 10, FIG. 11, FIG. 12, FIG. 13, FIG. 14, FIG. 15, FIG. 16, and FIG. 17) indicate that antagonism of the activity of TLR4 receptor complexes, such as by mab MTS510 treatment or decreasing TLR4 gene expression, can treat and prevent cartilage degradation in conditions such as osteoarthritis by preventing or decreasing the release of pro-inflammatory cytokines. In FIG. 6, FIG. 7, FIG. 8, FIG. 9, FIG. 10, FIG. 11, FIG. 12, FIG. 13, FIG. 14, FIG. 15, FIG. 16, and FIG. 17 the first four bars represent data from wild-type mouse articular cartilage explants; the remaining four bars represent data from TLR4 knock-out mouse articular cartilage explants. Data in FIG. 6, FIG. 7, FIG. 8, FIG. 9, FIG. 10, FIG. 11, FIG. 12, FIG. 13, FIG. 14, FIG. 15, FIG. 16, and FIG. 17 are presented as mean −/+standard deviation with N=4.

### EXAMPLE 4

### LPS Mediated Decreases in the Synthesis of the Articular Cartilage Component S-GAG by Human Chondrocytes Can Be Reversed By TLR4 Antagonist hTLR4-ECD Treatment

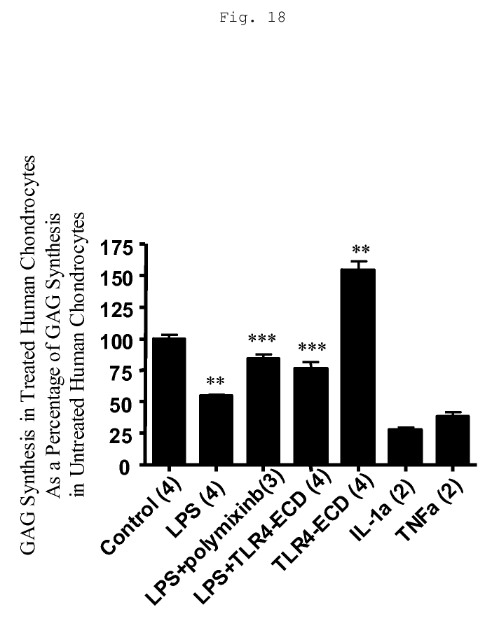

Lipopolysaccahride (LPS) mediated decreases in the synthesis of the articular cartilage component S-GAG in human chondrocytes can be reversed by treatment with the TLR4 antagonist hTLR4-ECD (FIG. 18). As seen in FIG. 18, the synthesis of the articular cartilage component S-GAG by human chondrocytes in alginate culture is decreased by LPS treatment. Importantly, treatment with hTLR4-ECD alone, or LPS and hTLR4-ECD resulted in increased S-GAG synthesis relative to chondrocytes treated with LPS alone (FIG. 18).

For these experiments, alginate cultures of human chondrocytes from healthy volunteers were supplied by Articular Engineering, LLC (Northbrook, Ill.) and were maintained as directed by the supplier. “Control” chondrocytes were untreated. “LPS” treated chondrocytes were treated with LPS at a concentration of 1 μg/ml in the media for three days starting at Day 2. “LPS+polymixin B” treated chondrocytes were treated for 1 hour with the LPS analog polymixin B, which is a TLR4 antagonist, at a concentration of 20 μg/ml followed by treatment with the TLR4 receptor agonist LPS at a concentration of 1 μg/ml in the media for three days starting at Day 2. “LPS+TLR4-ECD” treated chondrocytes were treated starting at Day 0 with the TLR4 antagonist TLR4-ECD at a concentration in the media of 50 μg/ml followed by treatment with LPS at a concentration of 1 μg/ml in the media for three days starting at Day 2. TLR4-ECD is a TLR4 receptor antagonist comprising the extracellular domain of hTLR4A and the Fc domain of an IgG1 isotype antibody. TLR4-ECD has the amino acid sequence shown in SEQ ID NO: 4 and is encoded by the cDNA having the nucleic acid sequence shown in SEQ ID NO: 3. TLR4-ECD preparations used here contained approximately 25% MD2. “TLR4-ECD” treated chondrocytes were treated starting at Day 0 with the TLR4 antagonist TLR4-ECD at a concentration in the media of 50 μg/ml. “IL-1alpha” (IL-1a) treated chondrocytes were treated with IL-1alpha at a concentration of 10 ng/ml in the media for three days starting at Day 2. “TNFalpha” (TNFa) treated chondrocytes were treated with TNFalpha at a concentration of 50 ng/ml in the media for three days starting at Day 2. On Day 5 alginate bead chondrocyte cell cultures were ³⁵S labelled overnight and ³⁵S incorporation into S-GAG (sulfated glycosaminoglycan) was then determined as described by Bobacz et al. 56 Arthritis. Rheum. 1880 (2007).

The results here (FIG. 18) indicate that antagonists of the activity of TLR4 receptor complexes, such as TLR4-ECD, can treat and prevent cartilage degradation in conditions such as osteoarthritis. Data in FIG. 18 are presented as mean −/+standard deviation with the number of cultures included in each treatment group indicated in parentheses after the X-axis descriptors. In FIG. 18, “**”=P<0.05 versus “Control” and P<0.001 versus “LPS.”

### EXAMPLE 5

### LPS Mediated Decreases in the Synthesis of the Articular Cartilage Component S-GAG by Human Chondrocytes from Osteoarthritis Patients Can Be Reversed By TLR4 Antagonist hTLR4 ECD Treatment

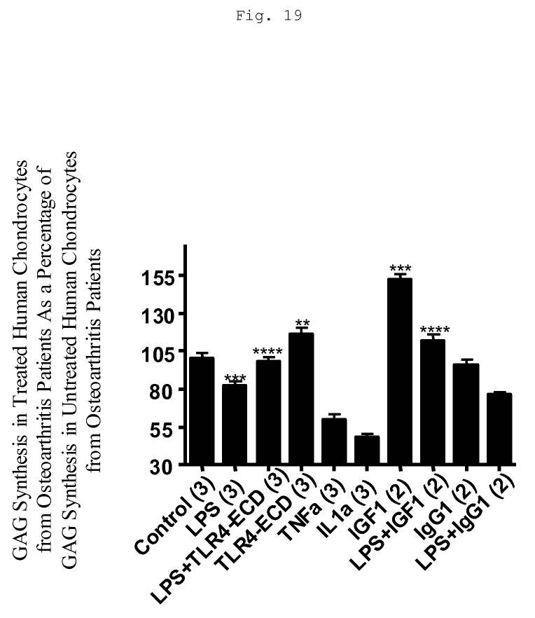

Lipopolysaccahride (LPS) mediated decreases in the synthesis of the articular cartilage component S-GAG in human chondrocytes from osteoarthritis patients can be reversed by treatment with the TLR4 antagonist hTLR4-ECD (FIG. 19). As seen in FIG. 19, the synthesis of the articular cartilage component S-GAG by human chondrocytes from osteoarthritis patients in alginate culture is decreased by LPS treatment. Importantly, treatment with hTLR4-ECD alone, or LPS and hTLR4-ECD resulted in increased S-GAG synthesis relative to chondrocytes from osteoarthritis patients treated with LPS alone (FIG. 19).

For these experiments, alginate cultures of human chondrocytes from osteoarthritis patients were supplied by Articular Engineering, LLC (Northbrook, Ill.) and were maintained as directed by the supplier. “Control,” “LPS,” “LPS+TLR4-ECD,” “TLR4-ECD,” “TNFalpha” (TNFa), and “IL-1alpha” (IL-1a) treatments of chondrocytes from osteoarthritis patients were as described in Example 4 above. “IGF1” (Insulin-like Growth Factor 1) treated chondrocytes were treated with IGF1 at a concentration of 100 ng/ml in the media for three days starting at Day 2. IGF1 is known to stimulate sGAG synthesis by human chondrocytes and was used as a positive control. “LPS+IGF1” treated chondrocytes were treated with IGF1 at a concentration of 100 ng/ml in the media starting and with LPS at a concentration of 1 μg/ml in the media for three days starting at Day 2. “IgG1” treated chondrocytes were treated starting at Day 0 with an IgG1 Fc domain at a concentration of 50 μg/ml in the media. “LPS+IgG1” treated chondrocytes were treated with IgG1 at a concentration of 50 μg/ml in the media for starting at Day 0 and were treated with LPS at a concentration of 1 μg/ml in the media for three days starting at Day 2. “IgG1” consists of an IgG1 Fc domain alone and was used a negative control to the Fc portion of the antagonist TLR4-ECD which comprises an Fc domain. At Day 5 alginate bead chondrocyte cell cultures were ³⁵S labelled overnight and ³⁵S incorporation into S-GAG (sulfated glycosaminoglycan) was then determined as described by Bobacz et al. 56 Arthritis. Rheum. 1880 (2007).

The results here (FIG. 19) indicate that antagonists of the activity of TLR4 receptor complexes, such as TLR4-ECD, can treat and prevent cartilage degradation in osteoarthritis patients. Data in FIG. 19 are presented as mean −/+standard deviation with the number of cultures included in each treatment group indicated in parentheses after the X-axis descriptors. In FIG. 19, “**”=P<0.05 versus “Control,” “***”=P<0.001 versus “Control,” and “****”=P<0.001 versus “LPS”; NC=Negative control.

The present invention now being fully described, it will be apparent to one of ordinary skill in the art that many changes and modifications can be made thereto without departing from the spirit or scope of the appended claims.

1. A method of treating an osteoarthritic condition comprising administering a therapeutically effective amount of a Toll Like Receptor 4 (TLR4) antagonist to a patient in need thereof for a time sufficient to treat the osteoarthritic condition.

2. The method of claim 1 wherein the TLR4 antagonist is an isolated antibody reactive with TLR4.

3. The method of claim 1 wherein the TLR4 antagonist is an isolated antibody reactive with TLR4 and having the antigen binding ability of the monoclonal antibody MTS510.

4. The method of claim 1 wherein the TLR4 antagonist comprises the extracellular domain of a TLR4.

5. The method of claim 4 wherein the TLR4 antagonist is a peptide chain comprising the amino acid sequence shown in SEQ ID NO: 4.

6. The method of claim 4 wherein the TLR4 antagonist is a peptide chain comprising the amino acid sequence shown in SEQ ID NO: 10.

7. A method of preventing an osteoarthritic condition comprising administering a therapeutically effective amount of a TLR4 antagonist to a patient in need thereof for a time sufficient to prevent the osteoarthritic condition.

8. The method of claim 7 wherein the TLR4 antagonist is an isolated antibody reactive with TLR4.

9. The method of claim 7 wherein the TLR4 antagonist is an isolated antibody reactive with TLR4 and having the antigen binding ability of the monoclonal antibody MTS510.

10. The method of claim 7 wherein the TLR4 antagonist comprises the extracellular domain of a TLR4.

11. The method of claim 10 wherein the TLR4 antagonist is a peptide chain comprising the amino acid sequence shown in SEQ ID NO: 4.

12. The method of claim 10 wherein the TLR4 antagonist is a peptide chain comprising the amino acid sequence shown in SEQ ID NO: 10.
